(19) United States
(12) Patent Application Publication
(10) Pub. No.: US 2008/0025874 A1
(43) Pub. Date: Jan. 31, 2008

(54) Method and apparatus for measuring and/or controlling the concentration of a gas in a solution

(76) Inventor: Michael F. Coughlin, Cincinnati, OH (US)

Correspondence Address: JohnsonDiversey, Inc., 8310 16TH STREET, M/S 509, PO BOX 902, STURTEVANT, WI, 53177-0902, US

(21) Appl. No.: 11/493,986

(22) Filed: Jul. 27, 2006

(51) Int. Cl. G01N 33/48

(52) U.S. Cl. 422/82.01; 422/68.1

(57) ABSTRACT

A device and method for measuring the concentration of a water soluble gas within an aqueous solution. In some embodiments, the solution has suspended solids, color, and turbidity. The device includes a gas release device in communication with a gas monitoring device. The gas release device is configured to cause the gas within the aqueous solution to transfer from the solution to an ambient gas phase. A chamber captures the released ambient gas and directs the gas toward the gas monitoring device.

### BACKGROUND OF THE INVENTION

Water soluble gases are introduced to aqueous solutions for many reasons. For example, chlorine dioxide can be used in aqueous solutions for cleaning, disinfecting, sanitizing, and/or the like. For example, it can be used in carcass chillers, water treatment, clean-in-place systems, flume water, pulp processing, and the like. When using chlorine dioxide for these purposes, it is valuable to be able to measure and control the concentration of chlorine dioxide in the solution. Similarly, when other water soluble gases are introduced to a solution, it can be valuable to be able to measure the concentration of gas in the solution.

Many different devices can be used to directly measure chlorine dioxide and/or other gases in an aqueous solution. For example, probes can be used to directly measure chlorine dioxide in a solution via redox, selective ion, or amperometry. Furthermore, optical devices are also used to directly measure the amount of chlorine dioxide in a solution. However, these devices and methods of directly measuring the gas while in the solution are cumbersome and inherently inaccurate in their ability to measure the gas in the solution containing suspended solids, turbidity, or color. Specifically, the probes have pores that tend to plug in the presence of suspended solids, which render the probe response weakened or destroyed, and the optical devices generally cannot differentiate the gas from a changing background of color or turbidity.

Problems associated with measuring chlorine dioxide or other gases in solutions having suspended solids, turbidity, and/or color can be found in many applications. However, these problems are especially acute in fruit and vegetable flume water and chicken, beef, and other carcass chill water.

### SUMMARY OF THE INVENTION

The present invention relates to a device and method of measuring a gas, such as chlorine dioxide, ozone, oxygen, ammonia, peracetic acid, and the like, in a solution, such as water. The device and method are particularly useful when compared to the prior art when the solution contains suspended solids, turbidity, and/or color. However, the device and method disclosed herein are not limited to solutions having solids, turbidity, and/or color. Further, unlike the devices discussed above, the present invention measures the gas indirectly. In other words, the gas is not measured directly while it is in the solution. Rather, at least a portion of the gas is forced out of the solution and the concentration of the gas driven out of the solution is measured. Many different devices and methods can be used to cause the gas to depart from the solution. For example, a sample of solution can be heated, shaken, otherwise agitated or placed in a vacuum to cause separation of the gas from the solution. Additionally, the gas can be passively diffused through a membrane, such as a hydrophobic membrane, or the solution can be sprayed through a nozzle to cause separation of the gas from the solution.

One particular embodiment is directed toward a device for measuring the concentration of a water soluble gas, such as chlorine dioxide within an aqueous solution having suspended solids, color, and turbidity. The device includes a gas release device in communication with a water soluble gas monitoring device. The gas release device is configured to cause at least a portion of the water soluble gas within the aqueous solution to transfer from the solution to an ambient gas phase. A chamber captures the released ambient water soluble gas and directs the water soluble gas toward the gas monitoring device. In some embodiments, the gas release device includes a spray nozzle positioned and configured to spray the aqueous solution within the chamber to render the water soluble gas only partially soluble in the solution. One way of doing this is to use the spray nozzle to atomize the solution.

Another embodiment is directed toward a device for controlling the concentration of chlorine dioxide in an aqueous solution contained within a reservoir. The device includes a chlorine dioxide gas monitoring device, a controller coupled to the chlorine dioxide gas monitoring device, a source of chlorine dioxide coupled to the reservoir and selectively dispensed into the reservoir via the controller, and a gas release device in communication with the chlorine dioxide gas monitoring device. The gas release device is configured to at least temporarily receive a portion of the aqueous solution from the reservoir and to cause at least a portion of the chlorine dioxide within the portion of the aqueous solution to transfer from the solution to an ambient gas phase. The gas release device has a chamber adapted to capture the ambient gas phase of the chlorine dioxide and direct the chlorine dioxide toward the gas monitoring device. In some embodiments, the aqueous solution contains at least one of turbidity, color, and suspended solids, which may cause difficulties with conventional control devices.

Yet another embodiment is directed to a method of measuring the concentration of a water soluble gas, such as chlorine dioxide within a reservoir of an aqueous solution containing chlorine dioxide. In some embodiments, the aqueous solution contains at least one of turbidity, color, and suspended solids, which may cause difficulties with conventional methods of measuring the concentration. The method includes withdrawing a sample of the aqueous solution from reservoir and driving the water soluble gas from the sample of the aqueous solution. The method also includes directing the water soluble gas driven from the aqueous solution to a gas monitor and measuring the amount of water soluble gas driven from the aqueous solution with the gas monitor.

Further aspects of the present invention, together with the organization and operation thereof, will become apparent from the following detailed description of the invention when taken in conjunction with the accompanying drawings.

### BRIEF DESCRIPTION OF THE DRAWINGS

FIG. 1 is a schematic representation of one embodiment of the present invention.

FIG. 2 illustrates a gas separation or release device embodying aspects of the present invention. The gas separation device is shown coupled to a gas sensor or monitor.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limited. The use of “including,” “comprising,” or “having” and variations thereof herein is meant to encompass the items listed thereafter and equivalents thereof as well as additional items. The terms “mounted,” “connected,” and “coupled” are used broadly and encompass both direct and indirect mounting, connecting and coupling. Further, “connected” and “coupled” are not restricted to physical or mechanical connections or couplings, and can include electrical connections or couplings, whether direct or indirect. Finally, as described in subsequent paragraphs, the specific mechanical configurations illustrated in the drawings are intended to exemplify embodiments of the invention. Accordingly, other alternative mechanical configurations are possible, and fall within the spirit and scope of the present invention.

The present invention relates to a device and method of measuring a water soluble gas, such as chlorine dioxide in a solution, such as water. In one particular application, the device and method are particularly suited for measuring the gas in a solution having suspended solids, turbidity, and/or color. However, the device and method can be used to measure the concentration of gas contained in other solutions not having suspended solids, turbidity, and/or color.

Some embodiments of the present invention measure the gas indirectly by forcing the gas out of the solution and then measuring concentration of the gas driven out of the solution. In one particular embodiment, a gas release device is in communication with a gas monitoring device to indirectly measure the concentration of the gas in a solution. The gas release device is configured to cause the gas within the aqueous solution to transfer from the solution to an ambient gas phase. A chamber captures the released ambient gas and directs the released gas toward the gas monitoring device.

The devices and methods disclosed herein can be used to measure the concentration of many different water soluble gases. For example, the devices and methods disclosed herein can be used to measure chlorine dioxide, ozone, oxygen, ammonia, peracetic acid, and many other water soluble gases. Although the remaining disclosure will provide one or more examples referencing a specific gas, such as chlorine dioxide, the examples should not be construed to be limited for use with that type of gas only.

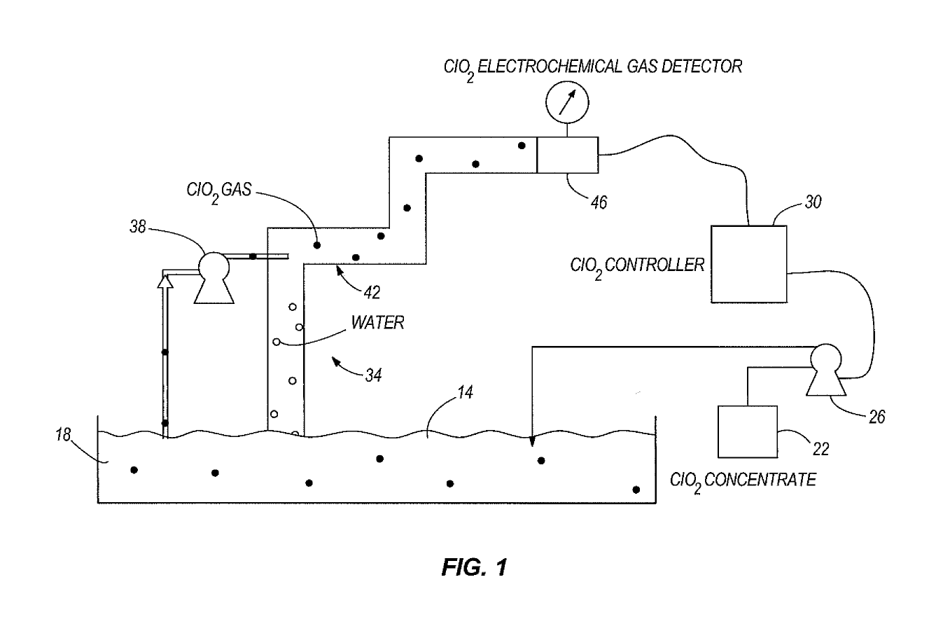

A schematic representation of one particular embodiment of the present invention is shown in FIG. 1. This figure illustrates one particular embodiment of a device 34 for releasing a gas from an aqueous mixture and measuring the released gas. Additionally, as will be described in greater detail below, this embodiment also includes controller coupled to a reservoir of treatment chemical. Upon measuring the concentration of the released gas, the controller can determine whether the measurement is below a threshold value, and if so, add gas or treatment chemical to the solution. Although the illustrated embodiment shows the controller and gas (or treatment chemical) source (note that the gas source can be an aqueous solution containing the gas), not all embodiments need to incorporate these features. For example, in some embodiments, the device can be used to merely release the gas from the solution.

In the illustrated embodiment of FIG. 1, a solution 14 is stored within a reservoir, tank, channel, sump, or other chamber 18. The solution 14 can have suspended solids, turbidity, and/or color due to its use with a meat chiller, such as a poultry chiller, a water treatment tank, part of a fruit and vegetable flume and wash water system, and the like. However, in other embodiments, the water may be substantially clear, due to its use for other purposes.

The solution 14 within the reservoir 18 also contains chlorine dioxide (or other water soluble gas). A source 22 of chlorine dioxide is coupled to the reservoir 18 or is in communication with the solution 14. Chlorine dioxide from the source 22 of chlorine dioxide can be selectively added to the reservoir 18 to maintain the concentration of chlorine dioxide within the solution 14. As illustrated, a pump 26 can be used to deliver the chlorine dioxide to the solution 14. However, in other embodiments, other devices can be used to deliver the chlorine to the solution 14 within the reservoir 18, such as one or more valves controlling a pressurized source of chlorine dioxide.

A controller 30 is coupled to the pump 26 to selectively operate the pump to allow or cause chlorine dioxide to be added to the solution 14. The controller can be substantially any type of controller; however, a WebMaster controller (sold by Walchem Corp. of Holliston, Mass., USA) has been found to work well in some embodiments. The controller generally causes the pump 26 to operate when the concentration of chlorine dioxide within the cleaning solution drops below a predetermined threshold value. The predetermined threshold value can have a variety of values depending upon the application of the solution, the load on the solution, the temperature of the solution, and the like.

In this embodiment, the concentration of the chlorine dioxide within the solution is determined by causing chlorine dioxide to depart from the solution 14 and then measuring the concentration or amount of chlorine dioxide gas that departed from the solution 14. FIG. 1 illustrates one particular embodiment of a device 34 for releasing a gas from an aqueous mixture and measuring the released gas. As illustrated, the device 34 includes a pump that draws the solution 14 from the reservoir 18 and sprays the solution 14 into a chamber 42, vessel, conduit, reservoir, or the like. This action of spraying the solution 14 into the chamber 42 renders the chlorine dioxide only partially soluble in the solution 14 and causes the chlorine dioxide to depart from the solution significantly in proportion to the amount of chlorine dioxide originally present in the solution 14. However, the evolution of chlorine dioxide depends upon the temperature, pressure, and volume. The chamber 42 is in communication with the solution reservoir 18 or has a flow path that directs solution 14 back to the reservoir 18. The chamber 42 is also coupled to or in communication with a chlorine dioxide gas monitor 46. In one embodiment, the gas monitor 46 is an electrochemical gas detector. In some embodiments, the gas monitor 46 can be an iTX multi-gas monitor sold by Industrial Scientific Corp. of Oakdale, Pa., USA. In other embodiments, the gas monitor 46 can be an iTrans gas monitor and transmitter also sold by Industrial Scientific Corp. The iTrans can be used in combination with a controller, as described below, to further control the concentration of gas within a solution.

In the illustrated embodiment, the chamber 42 provides a convoluted or tortured pathway between the point where the solution is sprayed and the gas monitor 46. In this embodiment, the convoluted pathway is defined by a series of elbows which act as water/gas baffle plates. However, in other embodiments, other devices can be used to impede the solution from reaching the gas monitor 46.

The gas monitor 46 is coupled to the chlorine dioxide controller 30. Accordingly, the gas monitor 46 communicates the sensed concentration of chlorine dioxide to the controller 30, which then can compare the sensed concentration to predetermined threshold levels. If the concentration is below the threshold value, the controller 30 causes additional chlorine dioxide to be added to the solution 14.

The device shown in FIG. 1 operates as follows. The pump 38 draws solution 14 from the reservoir 18 and expels it into the chamber 42. In some embodiments, the solution 14 is sprayed through a nozzle (not shown) to cause the solution to at least partially atomize. The spraying of the solution 14 causes the chlorine dioxide to depart from the solution 14 substantially in proportion to the concentration originally dissolved in the solution 14. The released chlorine dioxide gas travels through the pathway of the chamber 42 to the gas monitor or sensor 46 where the concentration or amount of chlorine dioxide released from the solution 14 is measured. The remainder of the sprayed solution 14 will flow through the chamber back toward the reservoir 18.

The measured concentration of chlorine dioxide is communicated to the controller 30. The controller compares the measured concentration to predetermined threshold values. If the concentration is below the threshold value, the controller causes additional chlorine dioxide to be added to the solution 14. In the illustrated embodiment, the pump 26 operates to deliver the chlorine dioxide to the solution 14. The controller can cause a predetermined amount of chlorine dioxide to be delivered or it can cause the chlorine dioxide to be continuously delivered until the sensed amount of chlorine dioxide within the solution exceeds a threshold limit. The type of delivery and/or the amount of chlorine dioxide delivered can depend upon the interval at which the concentration is tested, the location at which chlorine dioxide is delivered to the solution, the load on the solution, and the like.

In other embodiments, other gas release devices can be used. For example, other gas release devices can include an impinger, a sonicator, a shaker, a mixer, a heater, a vacuum or Vacutainer® (trademark of Becton, Dickinson and Company), as well as other devices that result in the transfer of chlorine dioxide from solution into an ambient gas phase.

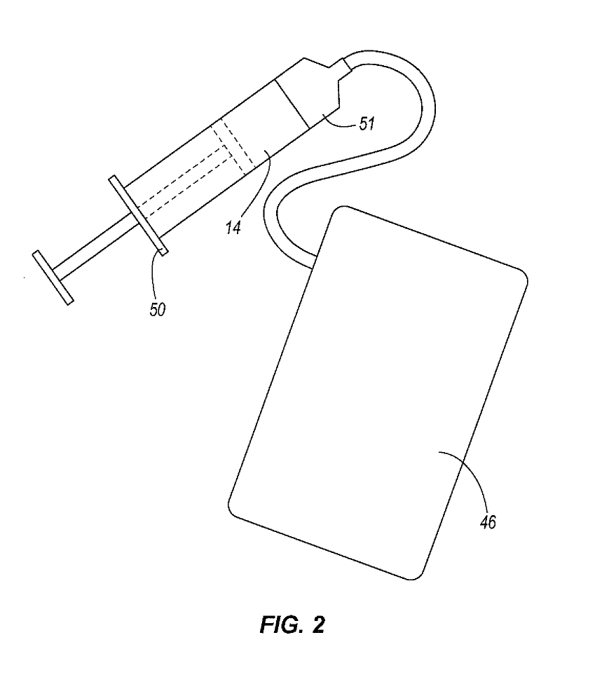

FIG. 2 illustrates one particular alternative embodiment of a gas separation device. As shown in this figure, a syringe 50 is used as the separation device. Specifically, a known volume of solution 14 or a sample can be drawn into the volumetric syringe to partially fill the syringe. The syringe can then be removed from fluid communication with the solution 14 within the reservoir 18 and the plunger of the syringe can be further drawn to add a known volume of air 51 to the syringe 50. Preferably, the air should not contain chlorine dioxide or should be substantially free from chlorine dioxide before it is drawn into the syringe. The syringe can then be shaken for a known, brief period of time to cause the chlorine dioxide to separate from the solution 14. The syringe can then be coupled to the gas monitor 46 and the plunger on the syringe can then be actuated to drive the air and the released chlorine dioxide to the gas monitor 46. Accordingly, the concentration of the chlorine dioxide can be measured.

Although a syringe is illustrated and described, other devices or combination of devices able to extract and hold known volumes of fluid can be used. For example, substantially any sealable container can be used to contain the solution and separate the chlorine dioxide. For example, the solution could be placed in a sealed test tube and shaken. Then, a syringe with a needle could be used to capture the released gas. Additionally, a sample can be placed in a container under vacuum (i.e., Vacutainer) and the vacuum can cause the gas to release from the solution.

In the embodiment shown in FIG. 2, precision of air and solution volume, as well as shake time can impact the resultant measurement of chlorine dioxide. Specifically, as the volume of air within the syringe increases, the indicated concentration of chlorine dioxide can decrease. Conversely, as the amount of solution 14 increases, the indicated concentration of chlorine dioxide can increase. Furthermore, the shake time is important because too little time will not allow the gas to escape from the solution and too much time will allow the gas to re-enter the solution. Finally, with some gas monitoring equipment, the rate in which gas is discharged to the monitoring device is important. Specifically, flow rates greater than one liter per minute can lead to imprecise measurements with some devices. One particular useful set of experimental conditions are as follows:

Solution drawn into the syringe: 10 ml

Air drawn into the syringe: 50 ml

Shake time: 5-10 seconds

Air displacement from syringe: 10 ml/sec.

Other sets of conditions can be used as well. The accuracy of measurements under other conditions can be determined experimentally and converted if needed by a determined conversion factor.

Experimental results relating to the embodiment shown in FIG. 2 are described below in Table 1. The following procedure was followed to create a set of control data (i.e., test results in the absence of suspended solids, turbidity, or color). An aliquot of the ClO₂ concentrate was introduced into 100 ml of deionized water. A 10 ml aliquot of the ClO₂ mixture was tested using the HACH pocket colorimeter using the DPD method. A 10 ml aliquot of the ClO₂ mixture was aspirated by the 60 ml syringe. The syringe was held upright and 50 ml of air was drawn into the syringe. The contents of the syringe were shaken vigorously for 8 seconds. The outlet of the syringe was connected to the iTX via the cap and tubing. The barrel of the syringe was plunged as to displace the air from the syringe into the iTX at an approximate rate of 10 ml/sec. The peak reading of the iTX was recorded. Then the 10 ml aliquot left in the syringe was tested using the HACH pocket colorimeter using the DPD method.

Test results at various concentrations of chlorine dioxide are provided below:

TABLE 1
Deionized Water with Concentrated Chlorine Dioxide (about 500 ppm)

| | ClO2 in | ClO2 in | ClO2 in syringe |
|---|---|---|---|
| Volume (mL) of | DI Water | DI Water | atmosphere |
| ClO2 Concentrate | before shaking | after shaking | after shaking |
| added to the 100 mls | Hach DPD | Hach DPD | iTX |
| DI Water | Method | Method | peak reading |
| 0.01 | 0.03 | 0.00 | 0.33 |
| | 0.01 | 0.00 | 0.24 |
| 0.01 AVERAGE | 0.02 | 0.00 | 0.29 |
| 0.05 | 0.21 | 0.17 | 1.41 |
| | 0.20 | 0.16 | 1.76 |
| 0.05 AVERAGE | 0.21 | 0.17 | 1.59 |
| 0.10 | 0.48 | 0.42 | 2.45 |
| | 0.46 | 0.38 | 3.55 |
| 0.10 AVERAGE | 0.47 | 0.40 | 3.00 |
| 0.25 | 1.21 | 1.02 | 7.56 |
| | 1.24 | 1.00 | 8.28 |
| 0.25 AVERAGE | 1.23 | 1.01 | 7.92 |
| 0.30 | 1.38 | 1.21 | 8.58 |
| | 1.50 | 1.25 | 12.30 |
| 0.30 AVERAGE | 1.44 | 1.23 | 10.44 |
| 0.50 | 2.59 | 2.03 | 16.25 |
| | 2.50 | 2.02 | 19.20 |
| 0.50 AVERAGE | 2.55 | 2.03 | 17.73 |
| 1.00 | 5.12 | 4.22 | 38.63 |
| | 5.11 | 4.13 | 45.47 |
| 1.00 AVERAGE | 5.12 | 4.18 | 42.05 |

As illustrated above, the amount of chlorine dioxide detected by the iTX peak reading is generally proportional to concentration of chlorine dioxide originally in the water. For example, the average iTX peak reading for the 0.05 average chlorine dioxide solution is generally five times greater than the average iTX peak reading for the 0.01 average chlorine dioxide solution. This proportional relationship generally holds true for all of the test data.

This control data can then be used to determine the exact correlation between the iTX peak reading and the actual concentration in the solution. Specifically, a graph or equation can be used to correlate the measured chlorine dioxide in the air to the amount originally in the solution. The original amount of chlorine dioxide in the solution can be determined by using the HACH DPD method.

Additional data was produced for the embodiment shown in FIG. 2. However, this test was conducted with a solution having suspended solids, turbidity, and/or color. Specifically, tomato juice was added to a dioionized water solution similar to the control group. The following procedure was followed for this test:

An aliquot of the ClO₂ concentrate was introduced into 100 ml of 0.16% v/v tomato juice. A 10 ml aliquot of the ClO₂ and juice mixture was aspirated by the 60 ml syringe. The syringe was held upright and 50 ml of air was drawn into the syringe. The contents of the syringe were shaken vigorously for 8 seconds. The outlet of the syringe was connected to the iTX via the cap and tubing. The barrel of the syringe was plunged as to displace the air from the syringe into the iTX at an approximate rate of 10 ml/sec. The peak reading of the iTX was recorded. Then the 10 ml aliquot left in the syringe was tested using the HACH pocket colorimeter using the DPD method.

The results of this experiment are shown below in Table 2.

TABLE 2
Tomato Juice (0.16%) with Concentrated Chlorine Dioxide
(about 500 ppm)

| Volume (mL) of | | | ClO2 |
|---|---|---|---|
| ClO2 Concentrate | ClO2 in tomato | ClO2 in tomato | in syringe |
| added to the 100 mls | juice water | juice water after | atmosphere |
| containing | before shaking | shaking | after |
| 0.16% tomato | Hach DPD | Hach DPD | shaking iTX |
| juice | Method | Method | peak reading |
| 0.25 | 0.20 | 0.39 | 0.00 |
| | 0.18 | 0.36 | 0.09 |
| 0.25 AVERAGE | 0.19 | 0.38 | 0.05 |
| 0.30 | 0.38 | 0.38 | 0.39 |
| | 0.33 | 0.36 | 0.47 |
| 0.30 AVERAGE | 0.36 | 0.37 | 0.43 |
| 0.50 | 1.24 | 1.30 | 6.76 |
| | 1.28 | 1.34 | 6.97 |
| 0.50 AVERAGE | 1.26 | 1.32 | 6.87 |
| 1.00 | 4.87 | 2.83 | 20.46 |
| | 3.90 | 2.64 | 19.89 |
| 1.00 AVERAGE | 4.39 | 2.74 | 20.18 |

The test results shown in Table 2 illustrate how unreliable the Hach DPD method and other colorimeter methods can be in the presence of solids, color, and turbidity. For example, in the first set of results (i.e., 0.25), the Hach DPD method appears to indicate that the amount of chlorine dioxide in the solution increased after testing, which is erroneous. This appears to illustrate that the color of the solution or the solids in the solution are interfering with the reading.

Using the iTX peak reading data and the control data discussed above, the actual concentration of chlorine dioxide in the solution can be determined (or at least closely approximated). Specifically, the reading acquired in Table 2 can be inserted into a graph or equation prepared from the control data to determine the actual concentration (or a close approximation) in the solution.

The embodiments described above and illustrated in the figures are presented by way of example only and are not intended as a limitation upon the concepts and principles of the present invention. As such, it will be appreciated by one having ordinary skill in the art that various changes in the elements and their configuration and arrangement are possible without departing from the spirit and scope of the present invention. For example, various alternatives to the certain features and elements of the present invention are described with reference to specific embodiments of the present invention. With the exception of features, elements, and manners of operation that are mutually exclusive of or are inconsistent with each embodiment described above, it should be noted that the alternative features, elements, and manners of operation described with reference to one particular embodiment are applicable to the other embodiments.

Various features of the invention are set forth in the following claims.

What is claimed is:

1. A device for measuring the concentration of chlorine dioxide within an aqueous solution containing chlorine dioxide and at least one of suspended solids, turbidity, and color, the device comprising:
a chlorine dioxide gas monitoring device;
a gas release device in communication with the chlorine dioxide gas monitoring device, the gas release device configured to at least temporarily receive at least a portion of the aqueous solution and to cause the chlorine dioxide within the aqueous solution to transfer from the solution to an ambient gas phase, the gas release device having a chamber adapted to capture the ambient gas phase of the chlorine dioxide and direct the chlorine dioxide toward the gas monitoring device.

2. The device of claim 1, wherein the gas release device comprises a spray nozzle positioned and configured to spray the aqueous solution within the chamber to render the chlorine dioxide only sparingly soluble in the solution.

3. The device of claim 2, wherein the spray nozzle atomizes the solution.

4. The device of claim 1, further comprising a pump coupled to the spray nozzle.

5. The device of claim 1, wherein the gas release device comprises membrane allowing the chlorine dioxide to be passively diffused from the solution.

6. The device of claim 1, wherein the gas release device comprises an impinger, a sonicator, or a heat source to render the chlorine dioxide only sparingly soluble in the solution.

7. The device of claim 1, wherein the gas release device comprises a vacuum applied to the solution draw the chlorine dioxide from the solution.

8. The device of claim 1, wherein the gas release device comprises a selectively sealable container adapted to separate the chlorine dioxide from the solution.

9. The device of claim 8, wherein the sealable container is a syringe that is shaken to release the chlorine dioxide from the solution.

10. The device of claim 8, wherein the sealable container is a container placed under a vacuum to release the chlorine dioxide from the solution.

11. A device for controlling the concentration of chlorine dioxide in an aqueous solution contained within a reservoir, the device comprising:
a chlorine dioxide gas monitoring device;
a controller in communication with the chlorine dioxide gas monitoring device;
a source of chlorine dioxide coupled to the reservoir and selectively dispensed into the reservoir via control of the controller; and
a gas release device in communication with the chlorine dioxide gas monitoring device, the gas release device configured to at least temporarily receive a portion of the aqueous solution from the reservoir and to cause the chlorine dioxide within the portion of the aqueous solution to transfer from the solution to an ambient gas phase, the gas release device having a chamber adapted to capture the ambient gas phase of the chlorine dioxide and direct the chlorine dioxide toward the gas monitoring device.

10. The device of claim 11, wherein the controller compares the measured value of chlorine dioxide to a predetermined value, the controller causing chlorine dioxide to be dispensed into the solution in response to the measured value being below the predetermined value.

11. The device of claim 11, wherein the gas release device comprises a spray nozzle positioned and configured to spray the aqueous solution within the chamber to render the chlorine dioxide only sparingly soluble in the solution.

12. The device of claim 11, wherein the spray nozzle atomizes the solution.

13. The device of claim 11, further comprising a pump coupled to the spray nozzle.

14. The device of claim 11, wherein the gas release device comprises an impinger or a sonicator.

15. The device of claim 11, wherein the gas release device comprises a selectively sealable container adapted to separate the chlorine dioxide from the solution.

16. The device of claim 15, wherein the sealable container is a syringe having a known volume of solution and air that is shaken to separate the chlorine dioxide from the solution.

17. The device of claim 15, wherein the sealable container is a container placed under a vacuum to release the chlorine dioxide from the solution.

18. The device of claim 11, wherein the gas release device comprises a heat source to render the chlorine dioxide only sparingly soluble in the solution.

19. The device of claim 11, wherein the gas release device comprises a vacuum applied to the solution draw the chlorine dioxide from the solution.

20. The device of claim 11, wherein the aqueous solution containing the chlorine dioxide includes at least one of turbidity, color, and/or suspended solids.

21. A method of measuring the concentration of chlorine dioxide within a reservoir of an aqueous solution containing chlorine dioxide, the method comprising:
withdrawing a sample of the aqueous solution from reservoir;
liberating at least a portion of the chlorine dioxide from the sample of the aqueous solution;
directing the chlorine dioxide driven from the aqueous solution to a gas monitor; and
measuring the amount of chlorine dioxide driven from the aqueous solution with the gas monitor.

22. The method of claim 21, wherein withdrawing a sample of aqueous solution from the reservoir further comprises withdrawing a select volume of fluid from the reservoir with a syringe.

23. The method of claim 22, further comprising:
drawing a select volume of air into the syringe; and
shaking the syringe for a predetermined period of time to liberate at least a portion of the chlorine dioxide from the sample of the aqueous solution.

24. The method of claim 21, wherein withdrawing a sample of aqueous solution from the reservoir further comprises withdrawing a volume of aqueous solution from the reservoir with a pump.

25. The method of claim 21, wherein liberating at least a portion of the chlorine dioxide from the sample of aqueous solution further comprises spraying the sample of the aqueous solution into a chamber.

26. The method of claim 21, wherein liberating at least a portion of the chlorine dioxide from the sample of aqueous solution further comprises acting upon the sample of the aqueous solution with a sonicator or an impinger.

27. The method of claim 21, wherein liberating at least a portion of the chlorine dioxide from the sample of aqueous solution further comprises applying a vacuum the sample of the aqueous solution.

28. The method of claim 21, wherein liberating at least a portion of the chlorine dioxide from the sample of aqueous solution further comprises applying heat to the sample.

29. The method of claim 21, wherein liberating at least a portion of the chlorine dioxide from the sample of aqueous solution further comprises at least one of shaking, stirring, and agitating the sample.

30. The method of claim 21, wherein the aqueous solution containing the chlorine dioxide includes at least one of turbidity, color, and/or suspended solids.

31. A device for measuring the concentration of a water soluble gas within an aqueous solution containing the water soluble gas, the device comprising:
a gas monitoring device;
a gas release device in communication with the gas monitoring device, the gas release device configured to at least temporarily receive the aqueous solution and to cause at least a portion of the water soluble gas within the aqueous solution to transfer from the solution to an ambient gas phase, the gas release device having a chamber adapted to capture the ambient gas phase of the water soluble gas and direct the water soluble toward the gas monitoring device.

32. The device of claim 31, wherein the gas release device comprises a spray nozzle positioned and configured to spray the aqueous solution within the chamber to render the water soluble gas only sparingly soluble in the solution.

33. The device of claim 31, further comprising a pump coupled to the spray nozzle.

34. The device of claim 31, wherein the gas release device comprises an impinger or a sonicator.

35. The device of claim 31, wherein the gas release device comprises a heat source or a vacuum source applied to a sample of the solution.

36. The device of claim 31, wherein the gas release device comprises a selectively sealable container adapted to separate the water soluble gas from the solution.

37. The device of claim 36, wherein the sealable container is a syringe having a known volume of solution and air that is shaken to separate the chlorine dioxide from the solution.

38. The device of claim 36, wherein the sealable container is a container placed under vacuum to separate the chlorine dioxide from the solution.

39. The device of claim 31, wherein the water soluble gas is chlorine dioxide.

40. The device of claim 31, wherein the aqueous solution containing the water soluble gas includes at least one of turbidity, color, and/or suspended solids.

41. The device of claim 31, wherein the gas release device comprises membrane allowing the water soluble gas to be passively diffused from the aqueous solution.

42. A method of measuring the concentration of water soluble gas within a reservoir of an aqueous solution containing the water soluble gas, the method comprising:
withdrawing a sample of the aqueous solution from reservoir;
driving the water soluble gas from the sample of the aqueous solution;
directing the water soluble gas driven from the aqueous solution to a gas monitor; and
measuring the amount of water soluble gas driven from the aqueous solution with the gas monitor.

43. The method of claim 42, wherein withdrawing a sample of aqueous solution from the reservoir further comprises withdrawing a select volume of fluid from the reservoir with a syringe.

44. The method of claim 43, further comprising:
drawing a select volume of air into the syringe; and
shaking the syringe for a predetermined period of time to liberate the water solutable gas from the sample of the aqueous solution.

45. The method of claim 42, wherein withdrawing a sample of aqueous solution from the reservoir further comprises withdrawing a volume of aqueous solution from the reservoir with a pump.

45. The method of claim 42, wherein driving the water soluble gas from the sample of aqueous solution further comprises spraying the sample of the aqueous solution into a chamber.

46. The method of claim 42, wherein driving the water soluble gas from the sample of aqueous solution further comprises acting upon the sample of the aqueous solution with a sonicator or impinger.

47. The method of claim 42, wherein driving the water soluble gas from the sample of aqueous solution further comprises placing the sample within a container providing a vacuum.

48. The method of claim 42, wherein driving the water soluble gas from the sample of aqueous solution further comprises shaking, stirring, or applying heat to the sample.

49. The method of claim 42, wherein driving the water soluble gas from the sample of aqueous solution further comprises diffusing the water soluble gas from the solution through a membrane.

50. The method of claim 42, wherein the water soluble gas is chlorine dioxide.

51. The method of claim 42, wherein the aqueous solution containing the water soluble gas includes at least one of turbidity, color, and/or suspended solids.
